(19) United States
(12) Patent Application Publication
(10) Pub. No.: US 2003/0088366 A1
(43) Pub. Date: May 8, 2003

(54) Computational method for the design of screening libraries for superfamilies of molecular targets having therapeutic utility

(75) Inventors: John Saunders, San Diego, CA (US); Xiao Chuan Wang, San Diego, CA (US); Karine Lavrador Erb, San Diego, CA (US); Brian J. Murphy, San Diego, CA (US); R. Scott Struthers, Encinitas, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA

(21) Appl. No.: 10/193,744

(22) Filed: Jul. 11, 2002

Related U.S. Application Data
(60) Provisional application No. 60/305,439, filed on Jul. 13, 2001.

(51) Int. Cl. G01N 33/48; A01N 37/18; A01N 43/04

(52) U.S. Cl. 702/19; 514/2; 514/44

(57) ABSTRACT

Computational method for the design of a calculated drug space and for the use of such drug space to identify focused screening libraries for drug discovery, as well as drugs identified by the same.

### CROSS-REFERENCE TO RELATED APPLICATION

This application claims the benefit of U.S. Provisional Patent Application No. 60/305,439 filed Jul. 13, 2001, where this provisional application is incorporated herein by reference in its entirety.

### BACKGROUND OF THE INVENTION

1. Technical Field

This invention is directed to a computational method for the design of a calculated drug space and for the use of said drug space to identify focused screening libraries for drug discovery, as well as drugs identified by the same.

2. Description of the Related Art

Combinatorial chemistry is defined as rapid synthesis of small, medium or large collections of “drug-like” molecules organized into libraries and related by the method of synthesis and/or the scaffold. At first, companies developed methods to generate huge, random libraries (commonly hundreds of thousands of compounds per library) often prepared as poorly characterized mixtures with the expectation of finding highly potent molecules that would interact with specific molecular targets. However, only occasionally does this “shotgun” approach provide the desired outcome and it is both expensive and fundamentally inefficient since the process, of necessity, generates questionable data with significant noise (false-positive and negative screening hits), creates unspecified redundancy of molecular structure or generally ignores most of the screening data. Thus it does not allow the medicinal chemist to map out the target's binding site since the data cannot reliably be interpreted. As the medicinal chemists entered the field, they brought with them the insistence that libraries contain pure, drug-like molecules and that both medicinal chemistry intuition and computational analysis and design should lead to a maximally informative library without the inherent redundancy and unreliability of shotgun libraries. To distinguish this newer synthetic approach, where every molecule is carefully purified and characterized, the phrase “high-throughput parallel synthesis” (HT-PS) is used.

Designing a library which specifically addresses a molecular target requires that one be able to characterize known molecules which have a similar method of action and select compounds for synthesis which have similar physical properties to these known ligands, in ways which are relevant to these mechanisms of action, and hence would increase the probability that the synthesized compounds will also act as ligands. This requires the ability to computationally measure similarity of both known compounds, and of large numbers of compounds in virtual libraries (collections of molecules derived from multiple templates that are readily synthesizable). Furthermore, this measure of similarity must be related to the biological activity of the compounds, such that compounds which are calculated to be near one another have a higher probability of exhibiting biologically similar activities, rather than compounds which are calculated to be distant.

This problem, and the more general problem of measuring diversity of compound collections or selecting diverse sets of compounds, has been the subject of intense research in recent years. A unifying thread in these efforts is the concept of a chemistry, or property, space for drug-like molecules. A property space is defined by a set of molecular descriptors, with each unique descriptor constituting a dimension in the space. Both high-dimensional and low-dimensional spaces have been explored. A high-dimensional space is typically defined by “molecular fingerprints” which are bit strings, where each bit contains information about the presence or absence of a molecular feature, such as a specific substructure, or other molecular characteristic. The dimensionality of these spaces is equal to the length of the bit string. Low-dimensional spaces consist of a small number of molecular descriptors, typically 3 to 7. These descriptors may be derived from 2D properties (topology, hydrogen bonding patterns, atomic properties, molecular weight, log P, dipole moment, etc) or 3D properties such as pharmacophore data, interatomic distances, or molecular surfaces. Low dimensional spaces offer a number of advantages, including the ability to visualize populations of compounds and their suitability for cell-based algorithms for subset selection.

Metrics have been developed for definition of low-dimensional property spaces. One such metric is DiverseSolutions™ BCUT (J. Chem. Inf. Comput. Sci., 39, 28 (1999)). In essence, BCUT's are based on the highest and lowest eigen values of a matrix which is unique for each molecule. This matrix consists of atomic properties along the diagonal (such as charge, polarizability, hydrogen bond donor/acceptor ability), and connectivity or interatomic distances in the off-diagonal elements. Although the underlying physical basis of the receptor relevance of these metrics is not well understood, spaces defined from BCUT metrics have repeatedly been shown to cluster compounds possessing a given receptor binding activity, while distributing inactive compounds throughout a much larger volume of the space. A variety of other commercial software tools are available which can define and visualize chemistry spaces, and select sets of compounds either focused in a given region or distributed to maximize their diversity.

While significant advances have been made in this field, there is still a need in the art for improved computational methods for the design of drug space, as well as for the use of such space to identify focused screening libraries for drug discovery and drugs identified by the same. The present invention fulfils these needs and provides further related advantages.

### BRIEF SUMMARY OF THE INVENTION

Drug space can be defined using several methods and this concept has been used to design diverse libraries in screening campaigns. The current invention, instead of attempting to define all of drug space, defines a space unique for various superfamilies of molecular targets. The distinct advantage of this approach is that a highly focused and relatively small screening library may be designed and readily synthesized having a high probability of being enriched in molecules (“hits”) that interact with members of the given superfamily. Such “hits”, using an iterative approach, may be optimized to give the desired potency and selectivity for an individual target within the superfamily. This methodology requires only that representative members of the selected family are known to interact with certain molecules—the process is therefore of significant utility when trying to identify novel ligands for other members of the same family.

Alternatively, the technique can be focused directly upon a single molecular target and hence identifies molecules that have a high probability of interacting with the single selected target. This requires that molecules that interact with the selected molecular target are already known and the method is therefore useful in identifying novel molecules for the same target but from distinctly different chemical series.

Molecular targets that are of interest therapeutically may be divided into various superfamilies based on mechanism of action (enzymes), function and/or signaling apparatus (receptors) or macromolecular structure (DNA). Examples of such superfamilies include proteases, kinases, phosphatases, G-protein coupled receptors (GPCRs), nuclear receptors, growth factor receptors, voltage-gated ion channels and ligand-gated ion channels. Each of these superfamilies may be further sub-divided: for example proteases can be sub-classified as aspartyl, serine, cysteine and metallo-proteases. GPCRs, for which over 1000 gene products are already known, have been sub-divided into 5 major classes (A-E) and these may be again divided on the basis of the nature of the endogenous activating ligand. Examples of the latter include the monoamines (e.g., dopamine), acids (e.g., PGE2), peptides bearing an obligatory positive charge (e.g., GnRH, α-MSH) and peptides bearing an obligatory negative charge (e.g., angiotensin-II, endothelin).

A general procedure by which regions of drug space may be associated with a superfamily of molecular targets, a subdivision of such a superfamily or a single molecular target may include all or some of the following steps:

compile an electronic data base of drug and drug-like molecules, such as a list of proprietary in-house molecules and/or other available drug databases such as the World Drug Index, MDRR and/or CMC set of molecules;

apply a cell-based molecular diversity algorithm, such as the BCUT algorithm, to determine which, and how many, molecular descriptors (properties) maximally distinguish the full set of molecules;

arbitrarily divide each descriptor (axis) into a plurality of cells per axis (e.g., 5 axes and 10 cells per axis);

project into this defined space molecules (preferably all molecules) which are known to interact with the selected family of molecular targets to serve as the “training set” for that family; and

determine the coordinates of all cells occupied by the training set combined with every neighboring cell (e.g., for a 5-dimensional space having 5 axes, this will be 35−1=242 neighboring cells).

The resulting set of coordinates for each cell so identified defines the “space” of the selected family. New ligands for this set of receptors will fall into this defined “space”.

An embodiment of the invention is the concept of analyzing neighboring cells that, in the context of 5-dimensional space, requires that each occupied cell has 3⁵−1=242 neighbors. Empirically, this is important for the following reasons: [1] The objective of the screening library is to generate multiple hits against any target—with drug space for a specific receptor, superfamily, etc. so defined, subsequent more dense sampling focused on this space will lead to the nanomolar ligands required of drug candidates. Thus, while highly active molecules reside within a few cells or even a single cell, progressively less active, but still active, compounds are layered in neighboring cells. [2] Different levels of molecular target promiscuity may determine the volume of drug space for a given target—it is well established, for example, that the dopamine D4 receptor is highly promiscuous based on the ease with which antagonists can be made. [3] Compounds residing in one cell may lie at, or close to, the boundaries of that cell so that compounds in the nearest neighboring cell may actually be closer than if they were located at the extremities of the occupied cell.

When dealing with the database of all drug and drug-like molecules, the system will generate a set of descriptors for all of drug space and this set will be used for each superfamily. Such descriptors may include charge, dipole moment, H-bond acceptors, H-bond donors, polarisability, lipophilicity, molecular weight, partial atomic charges and the like. Typically it is found that between 3 and 7 descriptors afford definition of drug space, preferably 4 to 7 descriptors, more preferably 4-6 descriptors, and most preferably 5 descriptors. The quantity and identification of descriptors may be made by computer algorithm, or be human-derived, generally with the attempt to maximize the space covered by the compendium of drug and drug-like molecules. Once the space occupied by the family of targets has been identified, it is a simple matter of selecting for synthesis those molecules contained within a virtual library which fall into that space.

Another embodiment of the present invention provides dealing with a single molecular target for which ligands are already known, the space may be defined directly and the algorithm can select which descriptors (‘axes’) and the preferred number of descriptors for that target.

A further embodiment involves the use of the computer generated drug space for screening or evaluating existing compounds for determining biological activity of said molecule.

Still a further embodiment involves the use of the computer generated drug space for screening or evaluating virtual compounds for determining biological activity of said molecule.

These and other aspects of this invention will be evident upon reference to the following detailed description.

### BRIEF DESCRIPTION OF THE DRAWINGS

FIG. 1 illustrates four sub-groups of GPCR ligands (dark dots) mapped into three dimensions of BCUT-space vs. the NBI All Drugs (gray dots). A, the monoamine set; B, the non-peptide acid set; C, GPCR-PA⁻ set; D, GPCR-PA⁺ set.

FIG. 2 illustrates templates selected for virtual library enumeration (points of monomer attachment are via NH or COOH groups).

FIG. 3 illustrates distribution of “hits” for the High Throughput assays for both the “test” and “random” sets.

FIG. 4 illustrates representative dose-response curves of “hits” from the test set. Top Panel: competition curves for MC4 receptor; Middle Panel: a MCH-related receptor; Bottom Panel: GnRH receptor. Kᵢ values are derived from the IC₅₀ values of inhibition using the Cheng-Prusoff equation.

FIG. 5 illustrates structure-activity trends for MCH-R ligands.

### DETAILED DESCRIPTION OF THE INVENTION

Stimulation of receptors linked to G-protein activation represents a primary mechanism by which cells sense changes in their external environment and convey that information to the cytosol through various effector mechanisms. Historically, these G-protein coupled receptors (GPCRs) have represented a ‘gold mine’ for drug discovery and over 30% of currently approved medicines act as either agonists or antagonists at such sites. With some notable exceptions, most successes have been achieved by drugs interacting at receptors for the simpler ligands, particularly the monoamines; this invention allows for the development of new drugs by competing with the more complex peptide ligands.

GPCRs have been classified into five categories based on extensive phylogenetic studies as indicated in Table 1 and these have been further subdivided into sub-classes based on the level of protein sequence homology. Those receptors that are activated by peptide ligands do not neatly fall into one of these sub-categories, but are distributed within sub-classes A and B. The following examples focus on receptors within these sub-classes that are activated by positively charged peptide ligands (Table 2). The increased interest in GPCRs activated by endogenous peptides is a reflection of the growing number of gene sequences that have now been assigned to such receptors and also the belief that the design of non-peptide ligands may now be a tractable problem thereby overcoming the well-documented limitations of peptides themselves as drugs. In addition, many of the new orphan GPCRs may turn out to be opportunities for therapeutic intervention and those that are activated by peptides would naturally fall into this proposal.

TABLE 1
Classification of GPCR sub-families.

| Class and Name | | Sub-Class | Examples |
|---|---|---|---|
| A | Rhodopsin-like | I | Melanocortin |
| | | II | Noradrenalin |
| | | III | Endothelin |
| | | IV | Bradykinin |
| | | V | Chemokine - |
| | | VI | Melatonin |
| B | Secretin-like | I | Corticotropin |
| | | | releasing |
| | | | hormone |
| | | II | Parathyroid |
| | | III | Glucagon |
| | | IV | Latrotoxin |
| C | Metabotropic glutamate-like | I-IV | Glutamate (I) |
| | | | GABA-B (III) |
| D | Fungal pheromone | — | STE2 gene |
| | | | product |
| E | c-AMP receptors | — | cAR1 |
| Others: | many (e.g., ‘orphans’) | — | GPR 47, 57, 58 |

To date, strategies for the discovery of non-peptide ligands have included high-throughput screening of random, large compound collections and/or combinatorial libraries, progressive replacement of amide bonds in fragments of peptides thought to be critical for binding and the synthesis of novel templates putatively mimicking a presumed secondary structural feature such as a p-turn mimetic. While all of these approaches are valid and have had some notable successes, they do not offer a systematic approach for the family of receptors under review. The current invention introduces the concept that the ‘property space’ associated for ligands for GPCR subfamilies are definable and form only a small fragment of what is referred to as a ‘drug-like’ space.

TABLE 2
Selected GPCRs of therapeutic utility which show
a requirement for a basic residue for binding.

| | Receptor | Ligand Charge | Potential |
|---|---|---|---|
| Peptide/Protein Ligand | Class | Preference | Indications |
| Bradykinin | A-IV | Basic (R^{1,9}) | Inflammation; |
| | | | Pain |
| Bombesin/Neuromedin | A-III | Basic (H¹²) | Cancer |
| Calcitonin Gene Related | B-I | Basic (H¹⁰) | Hypertension |
| Peptide | | | |
| Chemokine | A-V | Basic | Inflammation, |
| | | | cancer |
| Corticotropin Releasing | B-I | Basic (R¹⁶, | Anxiety/Depres- |
| Factor (CRF) | | R³⁵) | sion; inflam- |
| | | | mation |
| FSH Glycoprotein | A-V | Basic | Contraception; |
| Hormone | | | Infertility |
| Galanin | A-V | Basic (H¹⁴) | Pain, Obesity, |
| | | | Alzheimer's |
| Growth Hormone Re- | B-III | Basic (R¹¹) | Short Stature; |
| leasing Hormone | | | frailty; burn |
| | | | healing |
| Growth Hormone | A-III | Basic | Short Stature; |
| Secretagogue (GHS) | | | frailty; burn |
| | | | healing |
| Gonadotropin Releasing | A-V | Basic (R⁸) | Cancer; |
| hormone | | | endometriosis; |
| | | | infertility |
| LH Glycoprotein | A-V | Basic | Infertility; Cancer |
| Hormone | | | |
| Melanocortin Receptors | A-I | Basic (R⁸) | Obesity |
| Melanin Concentrating | A-V | Basic | Obesity |
| Hormone | | | |
| Neuropeptide Y | A-III | Basic | Obesity |
| Opioid | A-V | Basic | Pain |
| | | (H₂N—Y¹) | |
| Orexin | A-III | Basic (R¹⁵)/ | Obesity, Hyper- |
| | | Neutral | tension, unknown |
| Somatostatin | A-V | Basic (K⁹) | Diabetes |
| Tachykinin/Substance- | A-III | Basic | Pain; |
| P/Neurokinin | | | inflammation |
| Thyrotropin Releasing | A-III | Basic (H²) | Central Hypo- |
| Hormone | | | thyroidism, |
| | | | Depression, |
| Vasopressin | A-V | Basic (R⁸)^{b} | Hypertension |
| Vasotocin | A-V | Basic (R⁸) | |
| Vasoactive Intestinal | B-III | Basic (K¹⁵) | Inflammation, |
| Peptide (VIP) | | | Stroke, Asthma |

Although based on minimal structural evidence, it has been widely assumed that GPCR proteins are characterized by a seven helical motif, wherein the helices successively traverse the lipid membrane of the cell (Biochemical Society Transactions, 57, 81 (1991), Ann. Rep. Med. Chem., 30, 291 (1992), QSAR and Mol. Modelling, 497 (1996)). Thus starting with an N-terminal domain which lies outside the cell, the protein C-terminus eventually ends up within the cytosol and the channel thus formed, together with the extracellular domains, is the putative binding site for many synthetic ligands, either agonists or antagonists, that are competitive with the endogenous receptor agonist. Most models, sometimes supported by mutational and/or computational studies but sometimes not, have relied upon hydropathy profiles to predict the putative transmembrane (TM) domains of the receptor. Such plots, while representing a ‘good start’, do not accurately predict such domains for the (bovine) rhodopsin protein, the details of which can now be seen from the recently published crystal structure (Science, 289, 739 (2000)). Briefly, the key findings concerning the helical domains are TM 1, 2, 3 and 6 are significantly longer than those predicted from hydropathy plots (30, 30, 33 and 31 compared to 25, 25, 20 and 24 respectively) suggesting that interpretation of the position of residues within putative helical regions will have to be revised.

Another observation is impact of the ubiquitous disulphide bridge (Cys¹¹⁰-Cys¹⁸⁷ in rhodopsin), one of several ‘fingerprints’ within the GPCR superfamily, between the second and third extracellular (EC) loops. In rhodopsin, Cys¹¹⁰ is close to the extracellular surface of TM3 and places the EC2 loop in such a position to severely impede access of ligands to the helical bundle. With the assumption that rhodopsin truly is an adequate homology model on which to base predictions for GPCRs, it is this molecular feature in particular that causes a reconsideration about the way in which small molecule ligands approach and then interact with their receptor. A current model, which may need to be revised, is that while there is considerable divergence in the way GPCR ligands may form the first interaction with the receptor (the ‘collision’ complex), most agree that a secondary event, with residues in the TM domain being critically involved, is responsible for receptor activation. Thus, following binding, there is believed (Chem. & Biol., 4, 239 (1997)) to be a mutual conformational reorganization of the receptor/ligand complex to reach the activated state of the receptor that can then activate the G-protein. For simple ligands, such as the monoamines, where the homologous receptor has a short N-terminus (NT), both binding steps are thought to involve the TM's. However, for more complex ligands, such as peptides represented by MCH, GNRH and CRF, there is considerable evidence that the initial binding event may require predominantly the NT domain in consort with EC-loops; only the activation step requires the TM region. Nevertheless, single and multiple point mutational data supports the view that small molecule antagonists for such peptide activated GPCRs use a binding sites that, at least in part, requires interactions within the helical bundle.

The most crucial feature that is distinctive for the various sub-families of GPCR is the pattern of charged residues contained within the putative transmembrane helical bundle, and is useful in predicting the type of synthetic ligand that may bind to a given receptor. In most cases, it defines the key electrostatic interaction between ligand and receptor as has been clearly identified for several sub classes. For example, the monamines have been shown (J. Biol. Chem., 263, 10267 (1988)) to interact with Asp-113 (β₂ numbering) located on helix III approximately 8 Å from the extracellular surface. Mutation to Ala or Asn causes greater than 10,000-fold reduction in binding both for agonists and antagonists although the receptor remains fully coupled. Similarly, Lys-199 (on helix V) of the AT₁ receptor is the important binding locus for angiotensin-II and non-peptide antagonists; here the functionality is reversed. Therefore, at the outset of a drug discovery program, it is useful to have the profile of charged residues in mind.

Over the past 4-5 years, there has been increasing success in the discovery (Drug Discovery Today, 80 (1999)) of small, non-peptide ligands for those receptors that have as their ligand the more complex peptides but, with only a few exceptions, the breakthroughs have been predominantly antagonists. It will be apparent that some of the therapeutic indications listed in Table 2 will be addressed by antagonists of specific receptors (e.g., GnRH antagonist for endometriosis and prostate cancer) while others will require an agonist approach (e.g., MC₄ agonist for obesity). Where there has been success in identifying agonists, this has been achieved mostly by serendipity—either screening hits or subtle changes to known antagonists. For example, a simple change of hydrogen to methyl in the AT₁ antagonist L 158809 produced a potent, partial agonist. Similarly, benzodiazepine-based CCK_{A} agonists evolved from the antagonist on going from N-methyl to N-isopropyl. Finally, stereochemical differences can influence functional activity and can determine agonist and antagonist behaviour.

One subset of the G-protein coupled receptor (GPCR) superfamily is that which is activated by a peptide carrying an obligatory positively charged residue (GPCR-PA⁺). This subclass is exemplified by receptors for melanocortins, GnRH, galanin, MCH, orexin, and chemokine receptors variously involved in eating disorders, reproductive disorders, pain, narcolepsy, obesity, and inflammation. A region of chemical property space enriched in GPCR-PA⁺ ligands was identified. This was used to design and synthesize a ‘test’ library of 2025 single, pure compounds to sample portions of this property space associated with GPCR-PA⁺ligands. This library was evaluated by high-throughput screening against three different receptors and found to be highly enriched in ligands (4.5 to 61-fold) compared to a control set of 2024 randomly selected compounds.

In order to delineate GPCR-PA⁺ property space as a region of property space occupied by all drugs, a database composed of 187 molecules active against GPCR-PA⁺ was constructed from data available in the literature and from Neurocrine's proprietary compound database. A five-dimensional chemical diversity space using BCUT metrics was developed which showed that these known ligands clustered to a defined and relatively small region of this space—approximately 7% of drug space itself defined by the volume occupied by 81,560 “drug-like” compounds. In order to evaluate the feasibility of building a chemical library that samples these GPCR-PA⁺ ligand-rich regions, 2025 compounds were selected on the basis of their location in this chemistry space, synthesized and their activity at three different PA⁺ receptors assessed by high-throughput screening. The hit rates from this focused library was high (0.4 to 6%) and significantly better than a control set of 2024 compounds randomly selected from Neurocrine's corporate collection (0.05-0.3%).

### EXAMPLE 1

### A Screening Library for GPCRs Activated by Positively Charged Peptides

An electronic list of all known drugs and drug-like molecules was compiled from available data bases such as the NBI proprietary collection, MDDR, WDI, the Merck Index and the like (“NBI-All Drugs”); in total in excess of 100,000 molecules and this was filtered to remove outliers (e.g., mw>800, rotatable bonds>24) and the resulting training set saved as a Structure Data file (‘drugs_training_set.sd’). Drugs_training_set.sd was imported into the Diverse Solutions algorithm that uses BCUT metrics to define drug (chemistry) space. Using the software default settings, all descriptors were calculated for each molecule with over 200 BCUT metrics (descriptors) being considered by the algorithm. In turn the system was asked to optimise chemistry space for 3, 4, 5, 6 and 7 descriptors (‘axes’) at a time and the best dimensional space and the descriptors themselves were selected which allowed the maximum number of cells to be occupied within the selected drug space; thus the drugs were widely distributed and maximally separated from each other. For drugs_training_set.sd, this was five dimensional space with the five axes being: H-bond donor, H-bond acceptor, charge, polarizability (lo) and polarizability (high). Typically, in order to record occupancy of various regions of this drug space, cell-based methods were employed and each of the five axes was divided into 10 bins resulting in the partitioning of the entire space into 100,000 (10⁵) individual cells. Comparing the location or index of the occupied cells was used to measure the diversity/similarity between compounds, or collections of compounds.

Next, chemistry space was computed for the sub-family of the G-protein coupled receptors (GPCRs) that are activated by peptide ligands having an obligatory requirement for a basic center that is protonated under physiological conditions. This subset is referred to as GPCR-PA⁺, PA⁺ being the designation for the activating ligand. In order to delineate this space as a portion of property space occupied by all drugs (see above), a database composed of around 187 molecules active against GPCR-PA⁺ was constructed from data available in the literature and from Neurocrine's proprietary compound database. This ‘training set’ for GPCR-PA⁺ space formulation—GPCR-PA⁺_training_set.sd—was subjected to the BCUT analysis detailed above using the 5 dimensional space already determined for drugs-training_set.sd and the compounds' position and their cell occupancy in the NBI All Drugs space are assigned. Those cells occupied by GPCR-PA⁺ ligands and their neighbor cells are used to define the GPCR-PA⁺ subspace. Only 7% of the NBI All Drugs space is needed to define the GPCR-PA⁺ subspace.

The ‘coordinates’ for GPCR-PA⁺ space are:

| | UNIVERSAL |
|---|---|
| AXIS (DIMENSION) | COORDINATES^{a} |
| Charge (low) | 4-7 |
| H-Bond Acceptor | 2-9 |
| H-Bond Donor | 4-8 |
| Polarizability (low) | 1-7 |
| Polarizability (high) | 3-8 |

^{a}Based on a 10 cell per axis grid, 10⁵ cells in total.

### EXAMPLE 2

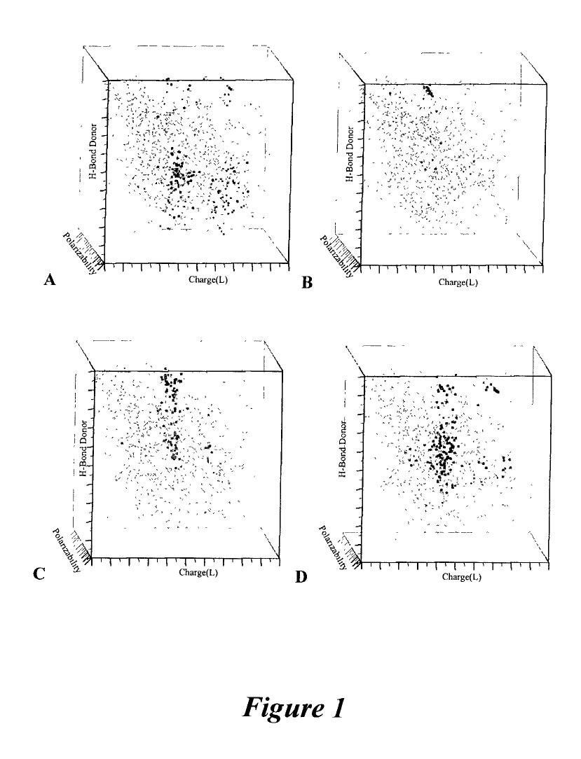

Ligands of four sub-groups of GPCRs represented within the initial training set—basic, presumably positively charged, ligands for the monoamine receptors, negatively charged non-peptide ligands (e.g prostanoid and lipid activated receptors) and, separately, positively and negatively charged ligands of peptide activated receptors were also compared. The locations of these compounds projected into 3 of the 5 dimensions of this diversity space is shown graphically in FIG. 1, and analysis of their cell occupancies in the full 5-dimensional space is presented in Table 3. The various GPCR ligand classes only occupy a small regions of this chemical space as compared to the space occupied by the original 81,560 compounds. This is reflected in Table 3 where the GPCR ligands occupy 397 cells, while the broader collection NBI All Drugs set occupies 8506 cells. All GPCR ligands occupy only a portion of drug space (19%); in addition, GPCR-PA⁺ forms a distinct sub-space (7%).

TABLE 3
Composition of the GPCR ‘training’, ‘test’ and random sets

| | | No. of | No. of | % Drug Space |
|---|---|---|---|---|
| | No. of | Cells | Occupied + | (NBI All |
| Receptor Class | Ligands | Occupied | Neighbor Cells | Drugs) |
| All Drugs + | 81,560 | 8506 | 91080 | 91 |
| NBI | | | | |
| (NBI-All | | | | |
| Drugs) | | | | |
| All GPCR | 630 | 397 | 19225 | 19 |
| Mono-amines | 201 | 165 | 8727 | 9 |
| Mono-acids | 35 | 34 | 2853 | 3 |
| GPCR-PA⁺ | 187 | 137 | 7255 | 7 |
| GPCR-PA⁻ | 106 | 79 | 4050 | 4 |
| Random set | 2024 | 1169 | 31098 | 31 |
| Test set | 2025 | 506 | 10692 | 11 |

### EXAMPLE 3

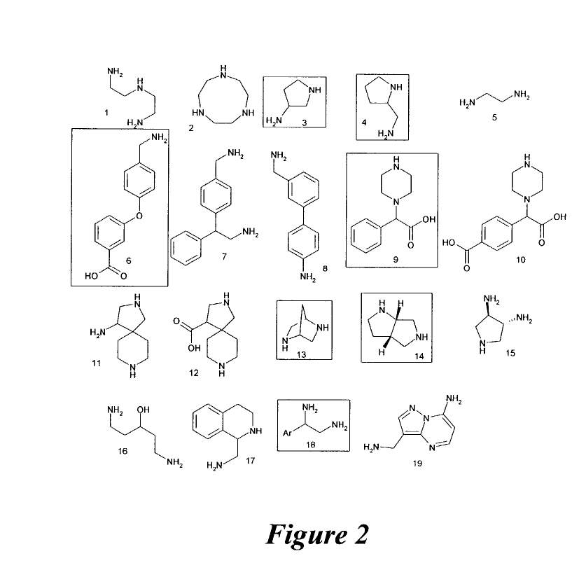

The five BCUT metrics discussed above were calculated for all compounds in the virtual libraries that had been enumerated electronically in order to evaluate the locations of the compounds relative to the GPCR-PA⁺ space defined above. Virtual libraries that had been selected for synthesis after approval by this design algorithm were first explored synthetically by generation of a small (typically 20 compounds) trial library. Albeit with only minimal experimentation, failure of chemistry at this early stage resulted in that template being (temporarily) rejected and a substitute template was then selected that closely mirrored the drug space of the former. From the 19 templates (FIG. 2) that were subjected to computational analysis, 10 virtual libraries were deemed to have compounds that fell into GPCR-PA⁺ space. Of these, 7 were able to be exploited with only minimal chemistry research (boxed in FIG. 2) and these form the basis of the 2025 member ‘test set’. From the 7 libraries with proven chemistry, synthetic libraries were designed from those compounds which lay within the target GPCR-PA⁺ region, but also which minimized the total number of advanced intermediates during the explosion phase described below in order to maximize the efficiency of the chemistry.

A parallel synthesis approach was used to generate the libraries by either solution-phase synthesis or polymer-supported synthesis. A core structure, orthogonally protected if required, named ‘template’, was first synthesized in a large scale (20 g). In all cases the template had at least two points of diversity. In cases with more than two points of diversity, ‘super-templates’ were first generated before the final step of library explosion. This process allowed final compounds to be generated in a matrix fashion and were purified by automated preparative high performance liquid chromatography that utilizes a mass spectrometer as a detector. All final compounds were synthesized on a scale that would produce at least 3 mg of material having greater than 85% purity and the correct molecular ion. Compounds that failed these criteria were rejected to avoid misinterpretation of biological data.

The design principle for the ‘test set’ required that a key feature be maintained in each molecule—here a basic nitrogen atom that will be predominantly protonated at physiological pH—the synthetic chemistry was restricted to reactions which preserved one or two such nitrogens of the original core or, alternatively, contained this feature in the reagents (‘monomers’) with which the core was reacted. As an example, consider template 3, which, in principle, can be subdivided into four sub-templates (Scheme 1). Noting that in some cases a protection strategy will be needed (BOC in this instance) to assure the correct regio-chemistry, acylation of the primary amine followed by alkylation at the secondary center will give access to 3a, reversing these steps, 3b, two sequential alkylations, 3c and two acylations, 3d the restriction being that 3d will carry a basic nitrogen in one of the two reagents used in the acylation reactions. For 3c, it was found that the second alkylation step was preferably conducted under reducing conditions employing an aldehyde monomer as the alkylating agent in the presence of sodium triacetoxyborohydride.

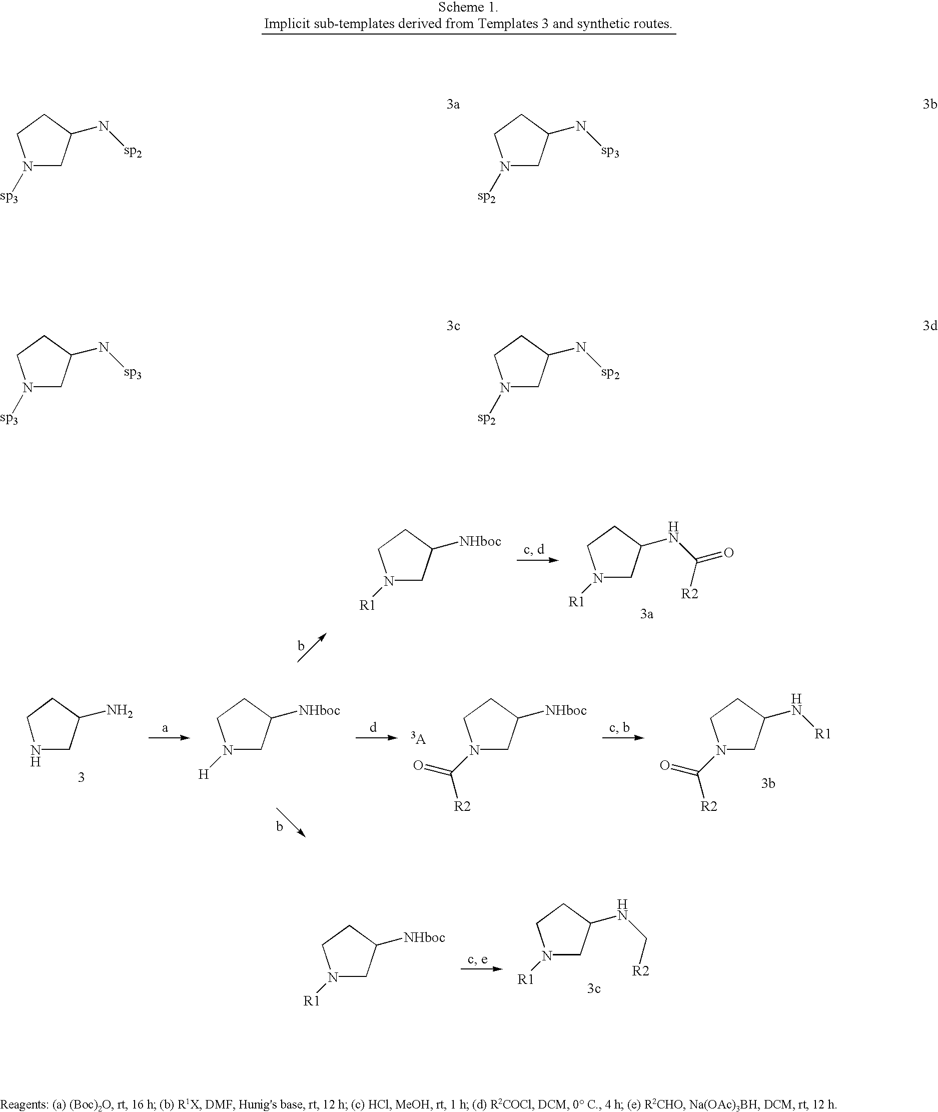

Template 4, also being a diamine like template 3 having a both a primary and secondary amine, may be considered as four implicit sub-templates as determined by the nature of the bonds formed upon library explosion (Scheme 2). The chemical reactions mirrored directly those developed for template 3 although it must be appreciated that the compounds so derived have distinctly different properties most notably the selected monomer inputs, the relative disposition of the two nitrogen atoms and the range of conformational degrees of freedom. In practice, only one sub-template (4a) was exemplified for the test set and, in this case, it was specifically the [S]-enantiomer that was used.

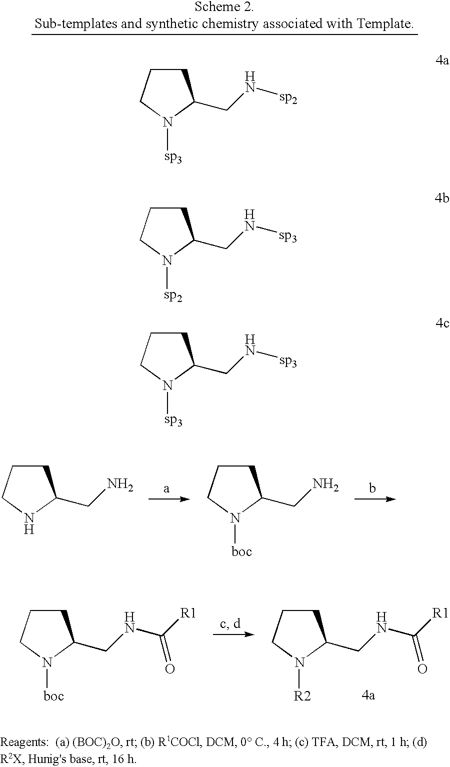

Exploitation of template 6 involved a combination of both solution and solid phase chemistry. The template was prepared in three steps from methyl 3-hydroxybenzoate and 4-fluorobenzonitrile as indicated (Scheme 3). The FMOC-protected amino acid was coupled to an amine charged polystyrene indole resin preloaded with the range of amines desired in the final library compounds. Deprotection of the coupled, now resin bound, intermediates followed by N-alkylation and cleavage from the resin with strong acid afforded the final library compounds.

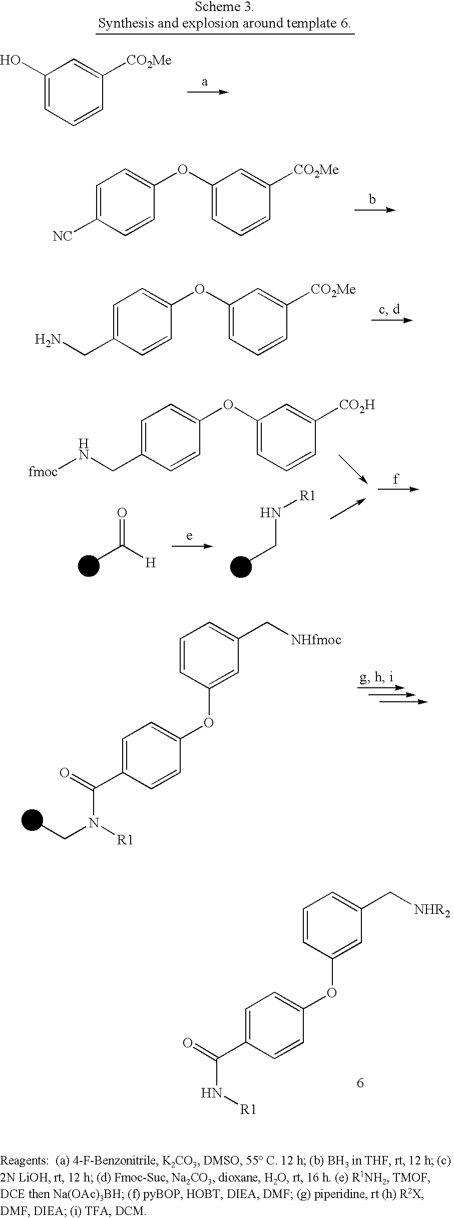

Aldehydes can react in a three component boronic acid-Mannich reaction to provide an expedient synthesis of amino acids as illustrated in the synthesis of Template 9a (Scheme 4). This reaction is based on simply mixing an aryl boronic acid, an amine (in this case mono-Boc-piperazine) and an aldehyde at room temperature. The resulting template after deprotection may then be decorated first by reductive amination with a range of aldehydes and then amidation of the carboxylic acid. As another sub-library (9b), wherein the basic center is migrated exo- to the piperazine ring, the alternative synthetic pathway may be followed. Here the unsubstituted piperazine-N was acylated with protected α-amino acids (BOC-glycine shown) and the subsequent steps required recapitulation of those described earlier for template 9a.

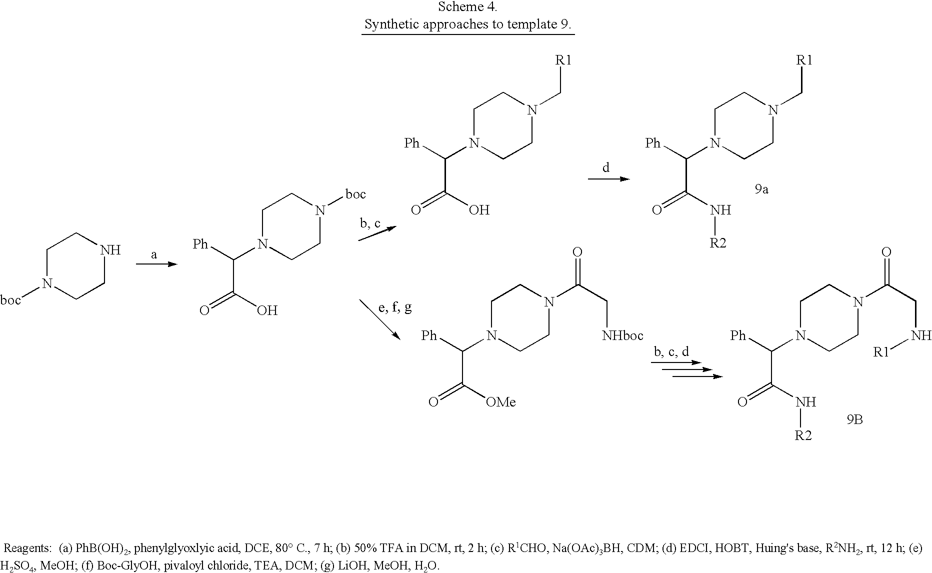

The 2,5-diazabicyclo[2.2.1]heptane template 13 represents a piperazine ring which has been forced into an energetically unfavourable boat conformation and as such induces a defined orientation of substituents in space distinctly different to piperazine. The symmetrical nature of the template restricts potential sub-templates to only two, 13a and 13b (Scheme 5). The free secondary amine was acylated with any acid or acid chloride (preferably acid) and after deprotection, an alkylation step afforded the required library 13a.

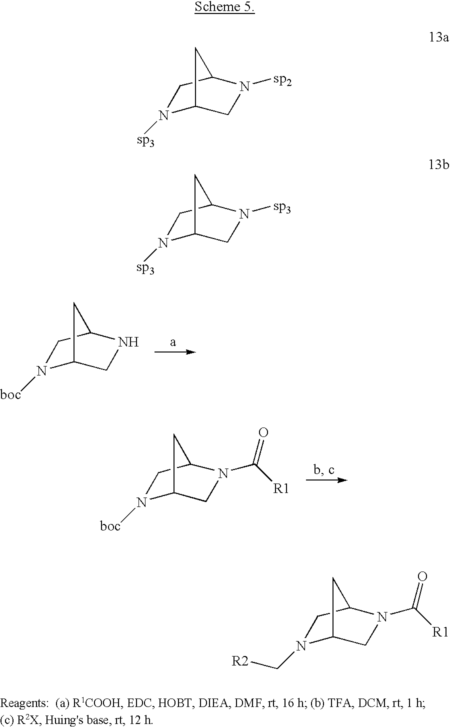

Intramolecular 1,3-dipolar cycloaddition of the azomethine ylide formed from a-carboxyiminium species (Scheme 6) gives access to the 2,7-diazabicyclo[3.3.0]octane ring of template 14. In turn these intermediates were obtained from a ketone bearing a pendant double bond in the side chain and α amino acids indicating that the diversity of potential starting templates is large. The resulting secondary amine was then acylated to yield the desired library compounds. By using a cyclic amino acid, a tricyclic ring was obtained which again was elaborated further by simple acylation.

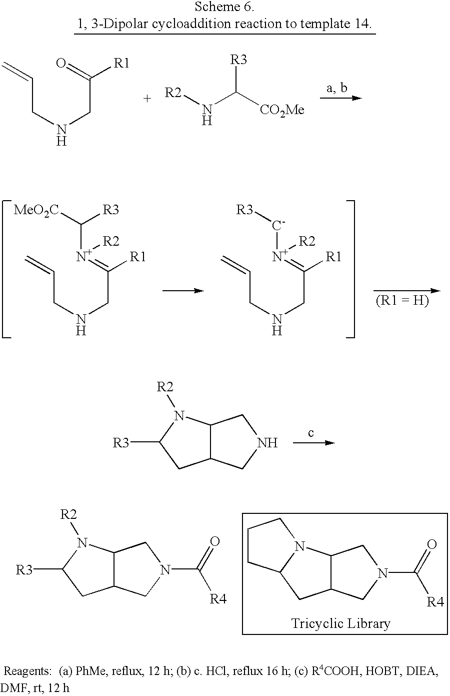

The more flexible template 18 can also be viewed as three distinct sub-templates (Scheme 7) and compounds relating to 18 can easily be obtained in two successive reductive alkylations. The first is a coupling of the diamine on Indole-Resin and the second employs a range of aldehydes to react with the resin bound diamine. The starting diamines were readily available by opening the relevant epoxide ring with an amine followed by activation of the resulting alcohol and a second displacement, this time using ammonia.

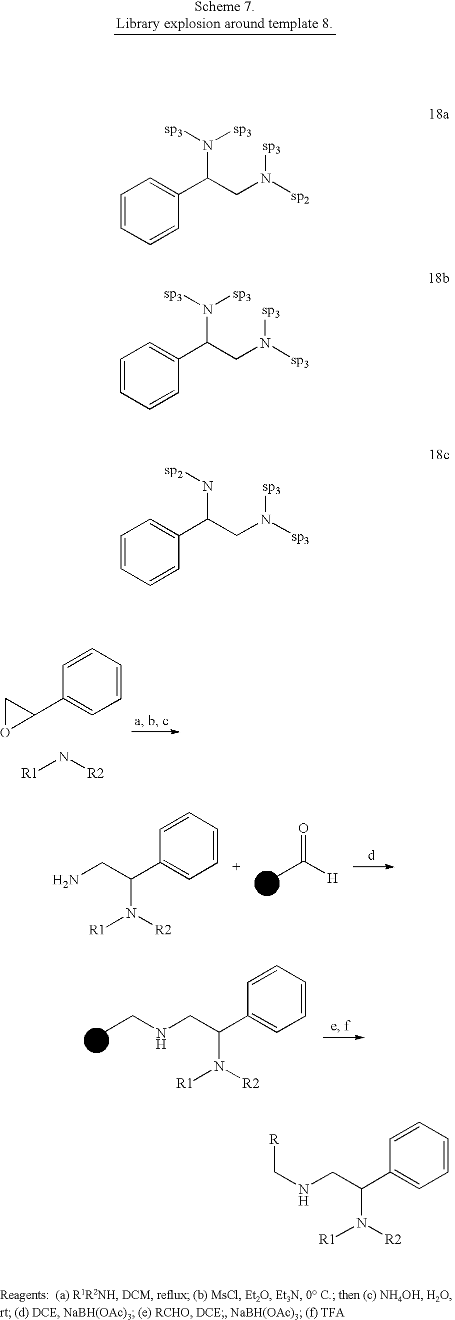

The 2025 compounds in the “test set” synthesized in the above reactions were arrayed in 96-well plates and were dissolved in DMSO to a standard concentration of 15 mM. A set of 2024 compounds selected randomly from Neurocrine's corporate screening collection was used as a control library. All compounds were then evaluated as a single group in three high throughput screens against receptors of high therapeutic potential. The melanocortin-4 receptor (MC4-R) is a potential target for the treatment of obesity and is a member of the A-I GPCR subclass. The melanin concentrating hormone receptor (MCH-R) has also recently been shown to be important in the control of feeding behavior and as such is also a potential drug target for the treatment of obesity. It is a member of the A-V subclass. The gonadotropin-releasing hormone receptor (GnRH-R) is a potential target for the treatment of endometriosis, uterine fibroids, prostate cancer and a range of other steroid hormone dependent diseases and is a member of the A-V subclass. Compounds were screened in radioligand binding assays that were terminated by rapid vacuum filtration. Compounds that displaced 50% or more of the specifically bound radioligand were confirmed by repeating them in duplicate.

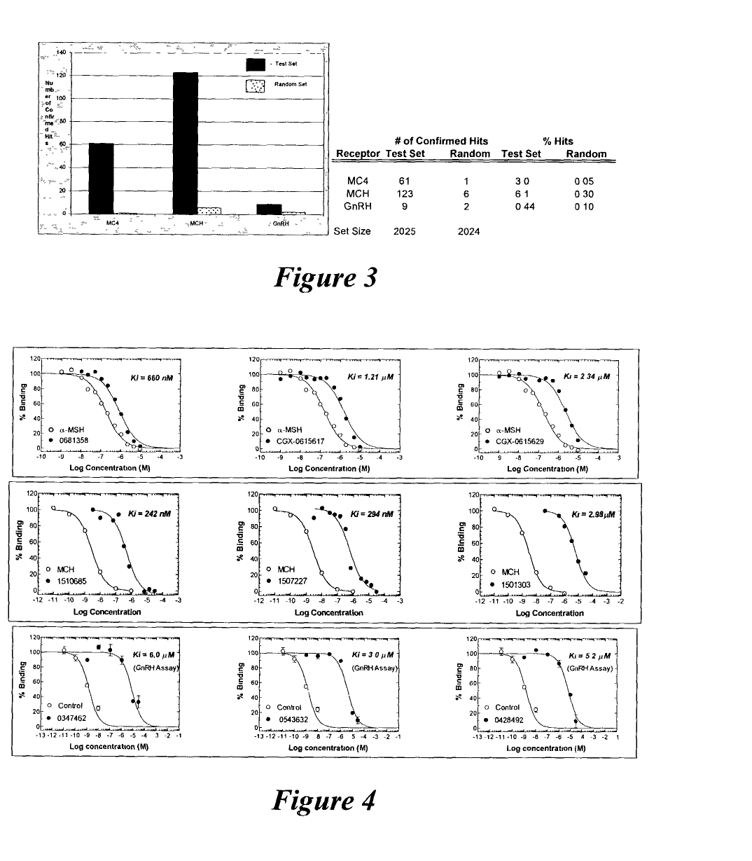

As an initial proof of concept for the design process, the number of hits identified in the designed set was significantly greater than those obtained from the random set for each of the three receptors (FIG. 3). Hit enrichment rates ranged from 4.5-fold (GnRH-R) to 61-fold (MC4-R). Thus, for the MC4 receptor the 2025 compound “test set” gave a number of hits which would have required screening more than 120,000 compounds of a typical corporate collection. The absolute number of hits varied from 9 for the GnRH receptor to 123 for the MCH receptor. In fact, when the MCH receptor was screened using the identical screening protocol against a 7140 compound library which had previously been selected by a committee of medicinal chemists based on their drug-like characteristics and presence of a positively charged nitrogen, only 41 hits (0.57% hit rate) were obtained. Thus, the use of the GPCR-PA⁺ chemistry space criteria resulted in a greater than 10-fold improvement in hit rates compared to a library carefully selected on the basis of chemical intuition.

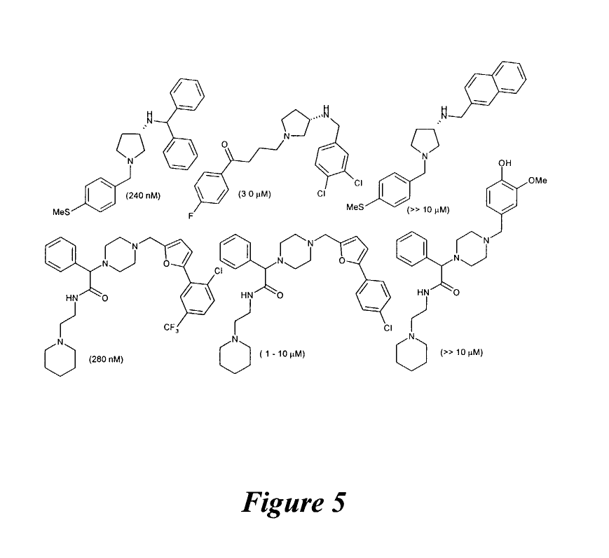

The differences in hit rate between the three receptors could either be due to intrinsic stringencies in the two receptors or to preferential sampling of MCH ligand enriched regions of property space in this initial subset of compounds. Analysis of the distribution of these hits in BCUT space suggests that a combination of both factors may be involved. The most active hits were titrated down with 12 point dose-response curves (representative curves shown in FIG. 4) and the Kᵢ determined for these compounds confirming that they reproducibly bind to the receptors studied with Kᵢ's in the range 240 nM-3 μM. Given that these molecules are close structural analogues of many others within the screening library which span the range of good activity (Kᵢ below 1 μM) through modestly active (Kᵢ below 10 μM) to inactive (<20% inhibition at 10 μM), this SAR data in itself represents an excellent starting point for a drug discovery project. The overall designed library adds yet another dimension however—actives are spread across more than one series of compounds (i.e., derived from more than one template). This facet strengthens the resulting computational model, the next step being to derive 3-D pharmacophores, and further increases the chance of being able to optimize activity below 10 nM following further iterations of design, synthesis and assay. As an example, for the MCH receptor, there are two prominent series in the active set (FIG. 5) and for each, three key structures that fall into the three activity levels are displayed.

It will be appreciated that, although specific embodiments of the invention have been described herein for purposes of illustration, various modifications may be made without departing from the spirit and scope of the invention. Accordingly, the invention is not limited except as by the appended claims.

1. A computer based method for generating a chemical space useful in the validation of potential molecular targets, which comprises the following steps:
compile an electronic data base of drug and drug-like molecules;
apply a cell-based molecular diversity algorithm to determine which, and how many, molecular descriptors (properties) maximally distinguish the full set of molecules;
arbitrarily divide each descriptor (axis) into a plurality of cells per axis;
project into this defined space molecules which are known to interact with the selected family of molecular targets to serve as the training set for that family; and
determine the coordinates of all cells occupied by the training set combined with every neighboring cell.

2. The method of claim 1 comprising the additional step of projecting a virtual molecule or set of virtual molecules into the defined chemical space and determining which virtual molecules fall into said defined space.

3. The method of claim 1 comprising the additional step of projecting an existing molecule or set of existing molecules into the defined chemical space and determining which existing molecules fall into said defined space.

4. The method of claim 1 wherein the selected family of molecular targets comprises drug target superfamilies.

5. The method of claim 1 wherein the selected family of molecular targets comprises GPCRs.

6. The method of claim 1 wherein the selected family of molecular targets comprises GPCR-PA⁺.

7. The method of claim 1 wherein the selected family of molecular targets comprises GPCR-PA⁻.

8. The method of claim 1 wherein the selected family of molecular targets comprises mono-amines.

9. The method of claim 1 wherein the selected family of molecular targets comprises mono-acids.

10. The method of claim 1 wherein the selected family of molecular targets comprises molecular targets for an individual receptor.

11. The method of claim 10 wherein the individual receptor is GnRH.

12. The method of claim 10 wherein the individual receptor is MC4.

13. The method of claim 10 wherein the individual receptor is MCH related.

14. A chemical space as defined in claim 1.

15. The chemical space of claims 14 wherein the selected family of molecular targets is GPCR-PA⁺.

16. A computer based method for generating a chemical space useful in the validation of potential molecular targets, which comprises the following steps:
compile an electronic data base of drug and drug-like molecules that interact with a single molecular target;
apply a cell-based molecular diversity algorithm to determine which, and how many, molecular descriptors (properties) maximally distinguish the full set of molecules;
arbitrarily divide each descriptor (‘axis’) into a plurality of cells per axis; and
determine the coordinates of all cells occupied by the training set combined with every neighboring cell.

17. The method of claim 16 comprising the additional step of projecting a virtual molecule or set of virtual molecules into the defined chemical space and determining which virtual molecules fall into said defined space.

18. The method of claim 16 comprising the additional step of projecting an existing molecule or set of existing molecules into the defined chemical space and determining which existing molecules fall into said defined space.

19. A drug or drug lead identified according to the method of any one of claims 1-13 and 16-18.
